(19) United States
(12) Patent Application Publication
(10) Pub. No.: US 2025/0128974 A1
(43) Pub. Date: Apr. 24, 2025

(54) TREATING SYSTEM FOR LIQUID WASTE CONTAINING ORGANIC MATTER

(71) Applicant: SHIMOSE MICROBES LABORATORY CORPORATION, Shimonoseki-shi (JP)

(72) Inventor: Shinichi SHIMOSE, Shimonoseki-shi (JP)

(21) Appl. No.: 19/000,829

(22) Filed: Dec. 24, 2024

Related U.S. Application Data
(63) Continuation of application No. PCT/JP2023/013026, filed on Mar. 29, 2023.

(30) Foreign Application Priority Data
Jun. 30, 2022 (JP) 2022-105700

(51) Int. Cl. C02F 3/28; C02F 103/20; C12M 1/00; C12M 1/06

(52) U.S. Cl. CPC C02F 3/28; C12M 27/02; C12M 29/04; C12M 29/06; C02F 2103/20

(57) ABSTRACT

The present invention provides a system for treating organic matter-containing liquid waste. The system is able to naturally dry a large amount of organic matter-containing liquid waste with reduced running costs and low capital investments. The system includes: a fermentation apparatus including a sealed container that stores a fermentation material in a storage and depressurizes and heats the storage, and a stirrer, and is configured to decompose organic components in the fermentation material using microorganisms; a treatment area in which a target plant P is placed; a transporter that transports a fermentation product S discharged from the fermentation apparatus after the fermentation of the fermentation material to the treatment area; and a discharger that discharges the fermentation product S from the transporter. The discharger is configured to discharge the fermentation product S toward the target plants P placed in the treatment area from above the target plants P.

### TECHNICAL FIELD

The present invention relates to a system for treating organic matter-containing liquid waste, for example, watery excrement of livestock such as dairy cows.

### BACKGROUND ART

The present inventor has invented a vacuum fermentation dryer as described in Patent Document 1, for example. In this dryer, excrement of livestock put in a sealed container such as a tank is efficiently dehydrated and dried by stirring the excrement under reduced pressure while heating to a predetermined temperature range. Further, predetermined microorganisms are added to the livestock excrement to be treated for accelerated fermentation of the excrement.

### CITATION LIST

### Patent Document

Patent Document 1: Japanese Unexamined Patent Publication No. 2020-65979

### SUMMARY OF THE INVENTION

### Technical Problem

However, it is difficult for the excrement treatment system described above to dry a large amount of watery excrement. Use of the dryer for a long time may increase running costs. Further, the system described in Patent Document 1 requires multiple vacuum fermentation dryers to treat a large amount of excrement and may require more capital investments.

The present invention has been made in view of the foregoing circumstances. An object of the invention is to provide a system for treating organic matter-containing liquid waste, the system being able to naturally fermenting and drying a large amount of organic matter-containing liquid waste with reduced running costs and low capital investments.

### Solution to the Problem

In order to achieve the object, the present invention disclosed in this specification is configured as follows. A first aspect of the present invention is directed to a system for treating organic matter-containing liquid waste. The system includes: a fermentation apparatus including a sealed container having a storage that stores a fermentation material which is organic matter-containing liquid waste, a storage pressure reducer that reduces pressure in the storage, a storage heater that heats the storage, and a stirrer having a stirring member that stirs the fermentation material in the storage, the fermentation apparatus being configured to decompose an organic component in the fermentation material using a microorganism and discharge a fermentation product produced from the fermentation material; a treatment area in which a target plant for making a bedding material is placed; a transporter that transports the fermentation product discharged from the fermentation apparatus to the treatment area; and a discharger that discharges the fermentation product from the transporter. The discharger is configured to discharge the fermentation product toward the target plant placed in the treatment area from above the target plant.

According to the first aspect of the present invention, fermenting the organic matter-containing liquid waste in the fermentation apparatus allows for production of a fermentation product rich in microorganisms which is useful for efficiently fermenting and drying the target plant. When the fermentation product is discharged to a large amount of target plant placed in the treatment area from above, the fermentation product is allowed to soak into the whole target plant while passing through the target plant from top to bottom by the action of gravity. This can provide a treatment system that can ferment and dry a large amount of organic matter-containing liquid waste with reduced running costs and low capital investments.

A second aspect of the present invention is an embodiment of the first aspect. In the second aspect, the treatment area includes a collection floor on which the target plant is placed. The collection floor collects a filtrate of the fermentation product that has passed through the target plant and flowed down from the target plant, and stores the filtrate in a storage tank.

According to the second aspect of the present invention, a liquid component of the fermentation product passes through the target plant and flows down from the target plant. The collection floor can thus accelerate the drying of the target plant. In addition, while the fermentation product passes through the target plant from top to bottom, a solid component in the fermentation product is caught by and left in the target plant. A filtrate obtained by passing the fermentation product through the target plant is collected toward the storage tank along the collection floor. Thus, the filtrate can be stored in the storage tank. The filtrate stored in the storage tank is allowed to flow through the target plant again from top to bottom, so that the filtrate can be fermented and dried up.

A third aspect of the present invention is an embodiment of the second aspect. In the third aspect, the collection floor has a first polygonal inclined plane and a second polygonal inclined plane, and the first polygonal inclined plane has a shared side that is shared with the second polygonal inclined plane. The shared side is inclined with respect to a horizontal plane, and one end of the shared side is formed to be the lowest portion of the collection floor, and the storage tank is arranged near the lowest portion.

According to the third aspect of the present invention, when the target plant is placed somewhere on the collection floor, the filtrate passing through the target plant from top to bottom is once collected to the shared side of the first polygonal inclined plane and the second polygonal inclined plane. The filtrate collected to the shared side can be smoothly guided to the lowest portion of the collection floor to be stored in the storage tank. Thus, the drying of the target plant is accelerated if the target plant is placed at any position on the collection floor. This increases the flexibility in placing the target plant within the treatment area. Further, when the filtrate stored in the storage tank is allowed to flow through the target plant from top to bottom again, the filtrate can be fermented and dried up.

A fourth aspect of the present invention is an embodiment of the second or third aspect. The system further includes: a repeat-spray passage for spraying the filtrate stored in the storage tank toward the target plant from a spray nozzle above the target plant, and a liquid feed pump for feeding the filtrate from the storage tank to the spray nozzle is provided at some midpoint of the repeat-spray passage.

According to the fourth aspect of the present invention, the filtrate can be repeatedly passed through the target plant from top to bottom, and a larger amount of microorganisms contained in the filtrate can adhere to the target plant, thereby accelerating the fermentation and drying of the target plant.

### Advantages of the Invention

The system for treating the organic matter-containing liquid waste of the present invention can naturally ferment and dry a large amount of organic matter-containing liquid waste. Thus, the system for treating the organic matter-containing liquid waste can be provided with reduced running costs and low capital investments.

### BRIEF DESCRIPTION OF THE DRAWINGS

FIG. 1 is a schematic plan view of an overall of a system for treating organic matter-containing liquid waste according to an embodiment of the present invention.

FIG. 2 illustrates an overall configuration of the system for treating the organic matter-containing liquid waste according to the embodiment.

FIG. 3 schematically illustrates an overall schematic configuration of a fermentation apparatus of the system for treating the organic matter-containing liquid waste according to the present embodiment.

FIG. 4 illustrates the system for treating the organic matter-containing liquid waste of the present embodiment after the treatment of target plants.

Embodiments of the present invention will be described with reference to the drawings.

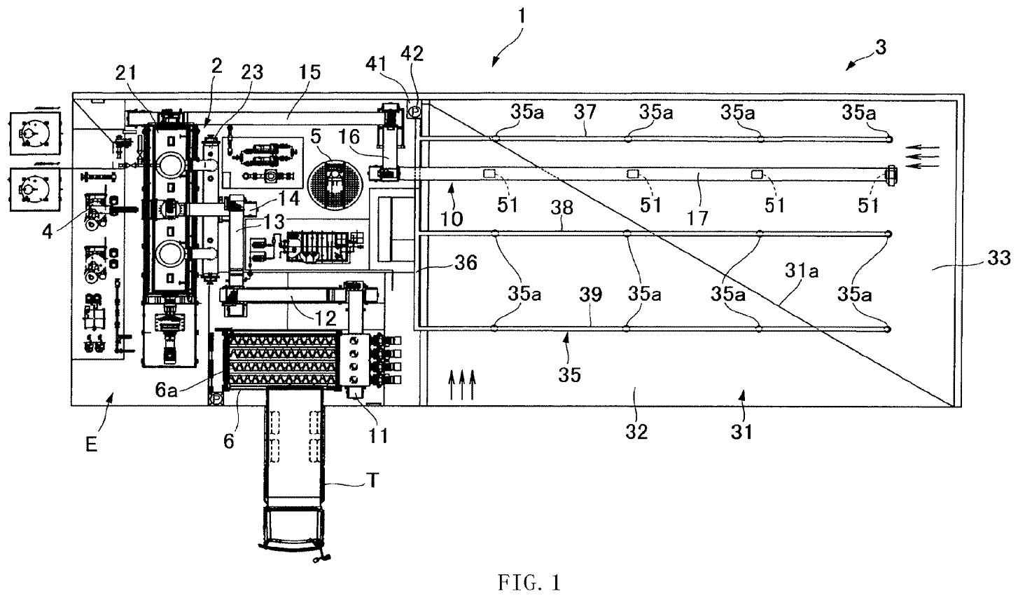

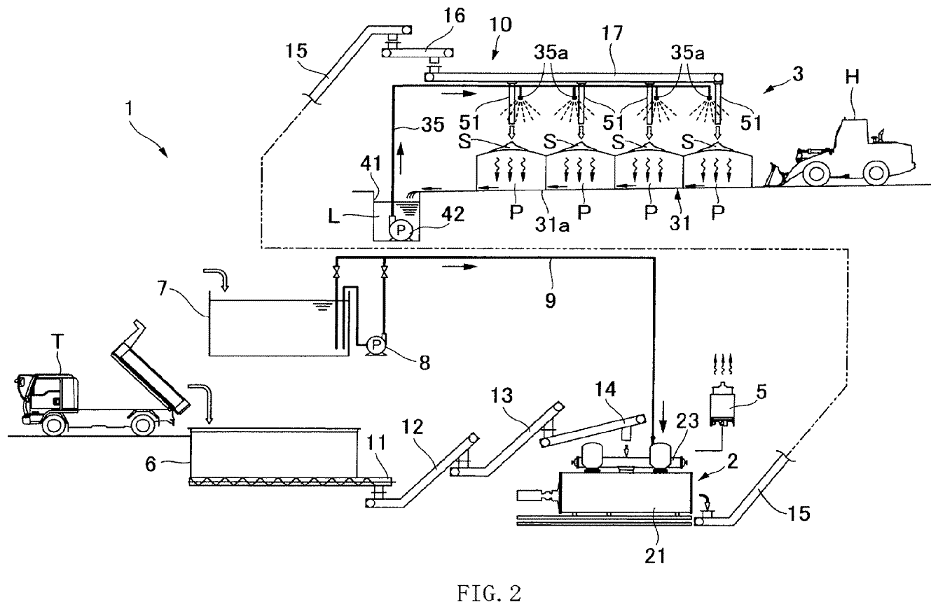

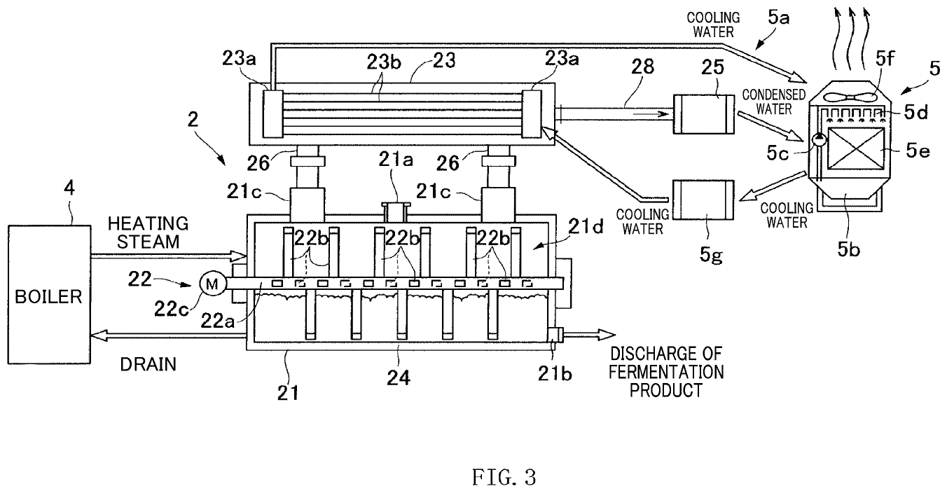

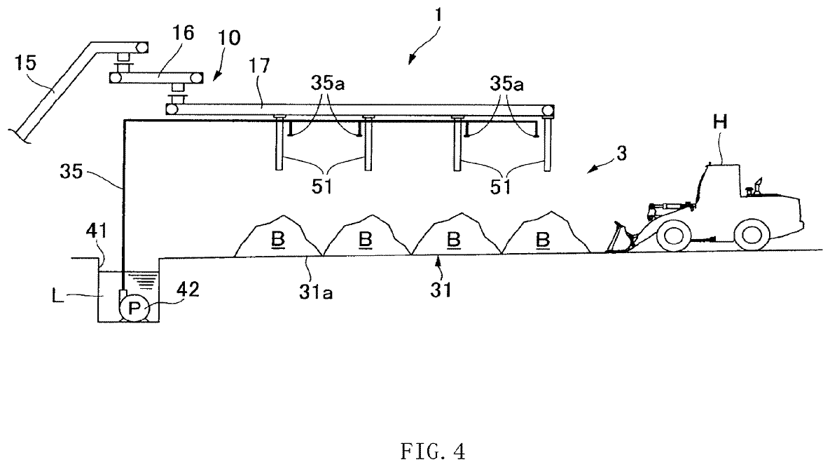

FIG. 1 is a schematic plan view of an overall of a system 1 for treating organic matter-containing liquid waste according to the present embodiment. FIG. 2 illustrates an overall configuration of the system 1 for treating the organic matter-containing liquid waste according to the present embodiment. FIG. 3 schematically illustrates an overall schematic configuration of a fermentation apparatus 2 of the system 1 for treating the organic matter-containing liquid waste according to the present embodiment. FIG. 4 illustrates the system 1 for treating the organic matter-containing liquid waste of the present embodiment after the treatment of target plants P. In FIGS. 1 to 3, the system 1 for treating the organic matter-containing liquid waste of the present embodiment includes a fermentation apparatus 2, a treatment area 3, a boiler 4, a cooling tower 5, a fermentation material feeding hopper 6, a liquid fermentation material feeder tank 7, a transporter 10, and a discharger 51.

The fermentation material feeding hopper 6 temporarily stores an excrement mixture of livestock such as dairy cows or organic sludge as a fermentation material. The excrement mixture is fed into the fermentation material feeding hopper 6 by a dump truck T carrying the mixture from, for example, a livestock barn. The target livestock includes, but is not limited to, dairy cows, pigs, sheep, and chickens, for example. The fermentation material feeding hopper 6 has a crushing screw 6a. The crushing screw 6a crushes large lumps of the fed excrement mixture into small pieces.

The fermentation material feeding hopper 6 and the fermentation apparatus 2 are connected by a first feeding conveyor 11, a second feeding conveyor 12, a third feeding conveyor 13, and a fourth feeding conveyor 14. The first feeding conveyor 11, the second feeding conveyor 12, the third feeding conveyor 13, and the fourth feeding conveyor 14 are parts of the transporter 10. These feeding conveyors feed slurry of the fermentation material from the fermentation material feeding hopper 6 to the fermentation apparatus 2. Each of the feeding conveyors transports the fermentation material linearly in plan view. The feeding conveyors are combined in the order of the first feeding conveyor 11, the second feeding conveyor 12, the third feeding conveyor 13, and the fourth feeding conveyor 14 from the fermentation material feeding hopper 6 so that the longitudinal directions of adjacent feeding conveyers are orthogonal to each other.

The liquid fermentation material feeder tank 7 temporarily stores, as a liquid fermentation material, urine with particularly low solid content in the excrement mixture of the livestock such as dairy cows. The liquid fermentation material feeder tank 7 and the fermentation apparatus 2 are connected by a liquid fermentation material feed pipe 9. A material feed pump 8 is provided at some midpoint of the liquid fermentation material feed pipe 9. The liquid fermentation material is supplied from the liquid fermentation material feeder tank 7 to the fermentation apparatus 2 by the action of the material feed pump 8.

The fermentation apparatus 2 is an apparatus for decomposing organic components in the fermentation material using microorganisms. As shown in FIG. 3, the fermentation apparatus 2 includes a sealed container 21, a stirrer 22, and a condenser 23.

As the sealed container 21, a substantially cylindrical pressure resistant tank is used, for example. The sealed container 21 stores the fermentation material, which is a mixture of livestock excrement or organic sludge, in a storage 21d. The storage 21d of the sealed container 21 is reduced in pressure by a storage pressure reducer to be described later and heated by a storage heater to be described later. The sealed container 21 is formed airtight to keep the storage 21d at atmospheric pressure or less. A heating jacket 24 is provided on a peripheral wall of the sealed container 21. The heating jacket 24 is connected to the boiler 4 by a heating steam supply passage which is not shown. Heating steam generated in the boiler 4 is supplied to the heating jacket 24. The heating jacket 24 and the boiler 4 constitute the storage heater.

A fermentation material inlet 21a is formed at the top of the sealed container 21. The fermentation material inlet 21a is connected to the fourth feeding conveyor 14. The fermentation material sent from the fermentation material feeding hopper 6 or the liquid fermentation material feeder tank 7 is put into the fermentation material inlet 21a. Guides 21c protrude from the top of the sealed container 21. The guides 21c are provided on one side and the other side in the longitudinal direction of the sealed container 21, respectively. The guides 21c guide vapor generated from the heated fermentation material to the condenser 23. A fermentation product outlet 21b for discharging a fermentation product S left after the fermentation of the fermentation material is formed in the bottom of a rear wall of the sealed container 21.

The stirrer 22 includes a stirring shaft 22a, stirring plates 22b, and an electric motor 22c. The stirring shaft 22a extends in the storage 21d of the sealed container 21 in the longitudinal direction of the sealed container 21. Multiple stirring plates 22b as a stirring member are provided on the stirring shaft 22a to be spaced apart from each other in the axial direction of the stirring shaft 22a. The electric motor 22c drives the stirring shaft 22a to rotate at a predetermined rotational speed.

The fermentation material put into the fermentation material inlet 21a is heated by the heat of the heating steam supplied to the heating jacket 24 while being stirred by the rotation of the stirring shaft 22a and the stirring plates 22b. The fermentation material, being heated and stirred, moves in the longitudinal direction of the sealed container 21 to be sent to the fermentation product outlet 21b.

Microorganisms are added to the fermentation material in the sealed container 21. Suitable examples of the microorganisms to be added include complex effective microorganisms obtained by culturing various types of autochthonous bacteria as a base in advance. That is, groups of so-called SHIMOSE 1, SHIMOSE 2 and SHIMOSE 3 have the majority of the colony. SHIMOSE 1 is FERM BP-7504 (deposited internationally on Mar. 14, 2003 in International Patent Organism depository, National Institute of Bioscience and Human-Technology, National Institute of Advanced Industrial Science and Technology, Ministry of Economic and Industrial Science and Technology, 1-3, Higashi 1-chome, Tsukuba, Ibaraki, Japan). SHIMOSE 2 is FERM BP-7505 (deposited internationally in the same manner as SHIMOSE 1), which is salt-tolerance microorganisms belonging to the genus Pichiafarinosa. SHIMOSE 3 is FERM BP-7506 (deposited internationally in the same manner as SHIMOSE 1), which is microorganisms belonging to the genus Staphylococcus.

The condenser 23 is connected to the guides 21c of the sealed container 21 by connecting pipes 26. The condenser 23 includes a plurality of cooling tubes 23b supported by a pair of heads 23a. Cooling water in the cooling tubes 23b increases in temperature due to heat exchange with the vapor entering the condenser 23 from the guides 21c. The cooling water in the cooling tubes 23b that has increased in temperature is cooled in the cooling tower 5. A cooling water passage 5a is provided between the cooling tubes 23b and the cooling tower 5.

The cooling tower 5 includes a water tank 5b, a pump 5c, a nozzle 5d, a downflow portion 5e, and a fan 5f. The cooling water discharged from the condenser 23 flows into the water tank 5b. The pump 5c pumps up the cooling water from the water tank 5b. The nozzle 5d sprays the pumped cooling water toward the downflow portion 5e. The fan 5f blows air toward the downflow portion 5e while the cooling water flows down in the downflow portion 5e. The cooling water is lowered in temperature by the air blowing from the fan 5f. The cooling water flows down through the downflow portion 5e, and then flows into the water tank 5b again.

The cooling water cooled by the cooling tower 5 is sent back to the condenser 23 by a cooling water pump 5g through the cooling water passage 5a. The cooling water sent back to the condenser 23 increases in temperature by exchanging heat with the vapor of the fermentation material while flowing through the cooling tubes 23b. The cooling water with the increased temperature passes through the cooling water passage 5a and flows into the cooling tower 5 again. That is, the cooling water circulates through the cooling water passage 5a between the condenser 23 and the cooling tower 5.

The vapor generated from the heated fermentation material is condensed in the condenser 23. In the cooling tower 5, condensed water generated in the condenser 23 also flows together with the cooling water circulating as described above. Specifically, a communication passage 28 is connected to the condenser 23. The condensed water generated in the condenser 23 stays in the condenser 23 and the communication passage 28. A vacuum pump 25 is connected to the communication passage 28. The vacuum pump 25 functions as the storage pressure reducer that reduces the pressure in the storage 21d of the sealed container 21. Thus, the vacuum pump 25 works to suck out the air and the condensed water from the condenser 23 via the communication passage 28. The vacuum pump 25 guides the air and the vapor in the storage 21d to the condenser 23 via the guides 21c and the connecting pipes 26. The condensed water sucked from the condenser 23 by the vacuum pump 25 is guided to the water tank 5b of the cooling tower 5 by a water pipe (not shown).

The condensed water guided to the water tank 5b of the cooling tower 5 is mixed with the cooling water and cooled. The condensed water contains the same microorganisms as those added to the fermentation material in the storage 21d, and odor components contained in the condensed water are decomposed by the microorganisms. Thus, odor contained in the condensed water is not emitted outside the sealed container 21.

The fermentation apparatus 2 is configured as described above. The fermentation material put in the fermentation material inlet 21a of the sealed container 21 is heated by the heating jacket 24 heated by the heating steam from the boiler 4. The pressure in the storage 21d of the sealed container 21 is reduced by the drive of the vacuum pump 25. The pressure reduction lowers the boiling point of water. Thus, the inside of the storage 21d is maintained at 50° C. to 60° C., and moisture contained in the fermentation material is boiled. The fermentation material is fermented more quickly when stirred by the stirring plates 22b of the stirrer 22.

As shown in FIG. 2, the fermentation product S after the fermentation of the fermentation material is sprayed on the target plants P such as wheat straw and sawdust so that the target plants P are fermented and dried for use as a bedding material. The fermentation product S is slurry rich in microorganisms. The fermentation product S can accelerate the fermentation of the wheat straw and the sawdust more than the fermentation material. Examples of the target plants P include, but are not limited to, the wheat straw and the sawdust. The target plants P are placed in a treatment area 3.

The treatment area 3 is a rectangular area in plan view as shown in FIG. 1. The treatment area 3 is next to an apparatus area E which is substantially square in plan view and includes components such as the fermentation apparatus 2, the boiler 4, the cooling tower 5, and the fermentation material feeding hopper 6. The treatment area 3 is in contact with the apparatus area E on one short side of the rectangle in plan view. The treatment area 3 has a collection floor 31 which is rectangular in plan view. The collection floor 31 has a first polygonal inclined plane 32 and a second polygonal inclined plane 33. Each of the polygonal inclined planes 32, 33 is a polygonal plane inclined with respect to the horizontal plane. The first polygonal inclined plane 32 and the second polygonal inclined plane 33 of the present embodiment are triangular planes. The first polygonal inclined plane 32 and the second polygonal inclined plane 33 are not limited to be triangular, and may be polygonal with four or more sides. The first polygonal inclined plane 32 has a shared side 31a shared with the second polygonal inclined plane 33. The shared side 31a is inclined with respect to the horizontal plane.

The shared side 31a runs along a diagonal line of the collection floor 31 which is rectangular in plan view. The shared side 31a is formed such that one end closer to the apparatus area E is the lowest portion of the collection floor 31 and the other end farther from the apparatus area E is the highest portion of the collection floor 31. The first polygonal inclined plane 32 and the second polygonal inclined plane 33 are each shaped to be inclined downward toward the shared side 31a. The highest portion of the collection floor 31 is formed in a peripheral portion of the collection floor 31 and has the same height as the floor of the apparatus area E.

A final discharging conveyor 17, which is linear in plan view, is arranged above the collection floor 31. The final discharging conveyor 17 is arranged parallel to the long sides of the collection floor 31 which is rectangular in plan view. The final discharging conveyor 17 enters the treatment area 3 from the apparatus area E and extends to be close to the end of the treatment area 3 opposite to the apparatus area E. The final discharging conveyor 17 is located at a predetermined height from the collection floor 31 by a plurality of pillars (not shown). The final discharging conveyor 17 and the fermentation apparatus 2 in the apparatus area E are connected by a first discharging conveyor 15 which is linear in plan view and a second discharging conveyor 16 which is linear in plan view. The first discharging conveyor 15, the second discharging conveyor 16, and the final discharging conveyor 17 are parts of the transporter 10. The first discharging conveyor 15, the second discharging conveyor 16, and the final discharging conveyor 17 transport the fermentation product S discharged from the fermentation apparatus 2 after the fermentation of the fermentation material to the treatment area 3.

As shown in FIGS. 1 and 2, multiple (four in the present embodiment) dischargers 51 are attached to the final discharging conveyor 17 and arranged at predetermined intervals. Each discharger 51 is formed of, for example, a flexible hose. The dischargers 51 are provided to hang down from the final discharging conveyor 17. The dischargers 51 discharge the fermentation product S from the final discharging conveyor 17. The target plants P are placed on the collection floor 31 of the treatment area 3 along the final discharging conveyor 17. The fermentation product S is discharged toward the target plants P placed on the collection floor 31 from above the target plants P.

A storage tank 41 is arranged near the lowest portion of the collection floor 31. The collection floor 31 collects a filtrate L of the fermentation product S that has passed through the target plants P and flowed down from the target plants P, and stores the filtrate L in the storage tank 41.

The system 1 for treating organic matter-containing liquid waste of the present embodiment includes a repeat-spray passage 35. The repeat-spray passage 35 is provided to repeatedly spray the filtrate L stored in the storage tank 41 toward the target plants P from above the target plants P. Specifically, the repeat-spray passage 35 includes a first pipe 36 extending along the boundary between the apparatus area E and the treatment area 3, and a second pipe 37, a third pipe 38, and a fourth pipe 39 that are branched from the first pipe 36. The second pipe 37, the third pipe 38, and the fourth pipe 39 are each arranged parallel to the long sides of the collection floor 31 which is rectangular in plan view and at a predetermined height from the collection floor 31. The second pipe 37, the third pipe 38, and the fourth pipe 39 extend to be close to the end of the treatment area 3 opposite to the apparatus area E. Each of the second pipe 37, the third pipe 38, and the fourth pipe 39 is provided with multiple spray nozzles 35a arranged at predetermined intervals.

A liquid feed pump 42 for feeding the filtrate L from the storage tank 41 to the spray nozzles 35a is provided at some midpoint of the repeat-spray passage 35. The filtrate L stored in the storage tank 41 is repeatedly sprayed toward the target plants P from the spray nozzles 35a above the target plants P.

In the treatment area 3 of the present embodiment, one final discharging conveyor 17 and three pipes, i.e., the second pipe 37, the third pipe 38, and the fourth pipe 39 that are branched from the first pipe 36, are arranged. The target plants P are arranged in the treatment area 3 along the final discharging conveyor 17, the second pipe 37, the third pipe 38, and the fourth pipe 39. The target plants P are placed using a wheel loader H.

When a new pile of target plants P is brought into the treatment area 3, the wheel loader H first places the new pile of target plants P along the final discharging conveyor 17. The slurry-like fermentation product S is discharged toward the target plants P from above the target plants P. After the fermentation product S is sufficiently soaked into the target plants P, the wheel loader H rearranges the target plants P along any of the second pipe 37, the third pipe 38, or the fourth pipe 39. The filtrate L of the fermentation product S is repeatedly sprayed on the target plants P from the spray nozzles 35a above the target plants P. The filtrate L of the fermentation product S is repeatedly sprayed on the target plants P for a first predetermined time. After the first predetermined time has passed, the target plant P is dried for a second predetermined time. Spraying the filtrate L on the target plants P for the first predetermined time and drying the target plants P for the second predetermined time are alternately repeated. Repeating this treatment allows efficient fermentation of the target plants P. Finally, as shown in FIG. 4, the target plants P that were hard before the treatment turn to a soft, fine, and high-quality bedding material B.

The disclosed embodiments are illustrative in all respects and do not serve as a basis for restrictive interpretation. The scope of the present invention is not interpreted only by the above-described embodiments, but is defined based on the description of the claims. The scope of the present invention includes all modifications within the meaning and scope equivalent to the scope of the claims.

### INDUSTRIAL APPLICABILITY

The present invention is applicable to a treatment system effective for organic matter-containing liquid waste, for example, watery excrement of livestock such as dairy cows.

### DESCRIPTION OF REFERENCE CHARACTERS

1 System For Treating Organic Matter-Containing Liquid Waste
2 Fermentation Apparatus
3 Treatment Area
10 Transporter
21 Sealed Container
21d Storage
22 Stirrer
22b Stirring Plate (Stirring Member)
31 Collection Floor
31a Shared Side
32 First Polygonal Inclined Plane
33 Second Polygonal Inclined Plane
35 Repeat-Spray Passage
35a Spray Nozzle
41 Storage Tank
42 Liquid Feed Pump
51 Discharger
L Filtrate
S Fermentation Product
P Target Plant

1. A system for treating organic matter-containing liquid waste, the system comprising:
a fermentation apparatus including a sealed container having a storage that stores the organic matter-containing liquid waste, a storage pressure reducer that reduces pressure in the storage, a storage heater that heats the storage, and a stirrer having a stirring member that stirs the organic matter-containing liquid waste in the storage, the fermentation apparatus being configured to decompose the organic matter-containing liquid waste using a microorganism and discharge a fermentation product produced from the organic matter-containing liquid waste;
a treatment area in which a target plant for making a bedding material is placed;
a transporter that transports the fermentation product discharged from the fermentation apparatus to the treatment area; and
a discharger that discharges the fermentation product from the transporter, wherein
the discharger is configured to discharge the fermentation product toward the target plant placed in the treatment area from above the target plant.

2. The system of claim 1, wherein
the treatment area includes a collection floor on which the target plant is placed, and
the collection floor collects a filtrate of the fermentation product that has passed through the target plant and flowed down from the target plant and stores the filtrate in a storage tank.

3. The system of claim 2, wherein
the collection floor has a first polygonal inclined plane and a second polygonal inclined plane,
the first polygonal inclined plane has a shared side that is shared with the second polygonal inclined plane,
the shared side is inclined with respect to a horizontal plane, and one end of the shared side is formed to be the lowest portion of the collection floor, and
the storage tank is arranged near the lowest portion.

4. The system of claim 2, further comprising:
a repeat-spray passage for spraying the filtrate stored in the storage tank toward the target plant from a spray nozzle above the target plant, wherein
a liquid feed pump for feeding the filtrate from the storage tank to the spray nozzle is provided at some midpoint of the repeat-spray passage.

5. The system of claim 3, further comprising:
a repeat-spray passage for spraying the filtrate stored in the storage tank toward the target plant from a spray nozzle above the target plant, wherein
a liquid feed pump for feeding the filtrate from the storage tank to the spray nozzle is provided at some midpoint of the repeat-spray passage.
